(19) United States
(12) Patent Application Publication
(10) Pub. No.: US 2026/0014312 A1
(43) Pub. Date: Jan. 15, 2026

(54) USER INTERFACE FOR PCA PUMP PROGRAMMING TO PREVENT DOSE ERRORS

(71) Applicants: Baxter International Inc., Deerfield, IL (US); BAXTER HEALTHCARE SA, Glattpark (Opfikon) (CH)

(72) Inventor: Rajesh Swarnkar, Bangalore (IN)

(21) Appl. No.: 19/263,021

(22) Filed: Jul. 8, 2025

(30) Foreign Application Priority Data
Jul. 12, 2024 (IN) 202441053379

(51) Int. Cl. A61M 5/145; A61M 5/142

(52) U.S. Cl. CPC A61M 5/1452; A61M 2005/14208; A61M 2202/0007; A61M 2202/0241; A61M 2205/18; A61M 2205/3327; A61M 2205/3382; A61M 2205/502

(57) ABSTRACT

Methods, systems, and apparatus are disclosed herein for a PCA pump user interface that provides on-screen parameter programming guidance to prevent dosage errors. The example methods, systems, and apparatus are configured to display parameter range line graphs on a PCA pump user interface screen in accordance with the lower and upper limits established for a user-inputted parameter. Additionally, the example methods, systems, and apparatus are configured to generate and display user alerts and does not allow users to enter the parameter if it is not in acceptable range when a user inputted parameter value affects the range of values acceptable by a previously entered parameter value.

### PRIORITY CLAIM AND CROSS-REFERENCE TO RELATED APPLICATIONS

The present application claims priority to and the benefit of Indian Provisional Patent Application No. 202441053379 filed Jul. 12, 2024, which is incorporated herein by reference in its entirety and relied upon.

### BACKGROUND

Patient Control Analgesia (PCA) pumps are commonly used for administering pain medication intravenously. PCA pumps are programmed to deliver an infusion at a continuous constant rate or can include Patient Control Analgesia (PCA) boluses, boluses like loading dose, clinician bolus, Patient Control Epidural Analgesia (PCEA) boluses, PIEB (Programmable Intermittent Epidural Bolus). To administer an infusion, the user programs the PCA pump. PCA pumps include multiple parameters to be programmed by the user, and each parameter has certain acceptable ranges (lower and upper limits). The parameters are interrelated, which makes programming more challenging and leads to programming errors that can delay therapy administration and lead to patient discomfort due to under dosing and overdosing. The user also enters lockout intervals defining the minimum time between consecutive PCA bolus administrations. Certain patient populations, like the opioid naïve, opioid tolerant and pediatric patients require extra PCA bolus monitoring, as they are susceptible to overdosing. PCA pumps also only display syringe volume at the start of a therapy but fail to ensure the programmed syringe volume and loaded syringe volume match.

Currently, PCA pumps display a user interface for programming each parameter individually. However, the user has to toggle between screens to program infusion therapies since the single screen does not display all the parameters together or the interrelation with the other parameters. As such, the user spends considerable time toggling between screens until the user can correctly program the infusion parameters for a given PCA therapy. Current PCA pumps also lack lockout interval safeguards, which can lead to over or under dosing. PCA pumps also lack the ability for the user to change the number of allowed PCA boluses from the calculated maximum, as needed for certain populations. The relationship between calculated number of PCA boluses when programming a PCA dose and lockout interval for a particular therapy, may not match with the number of PCA doses allowed for a patient.

A need accordingly exists for a PCA pump interface that provides on-screen programming guidance for multiple interrelated parameters, compares loaded and calculated syringe volume, calculates and displays lockout intervals, and allows the user to reduce the number of allowed PCA boluses.

### SUMMARY

Example systems, methods, and apparatus are disclosed herein for a PCA pump user interface that provides on-screen parameter programming guidance to prevent dosage errors. The example methods, systems, and apparatus are configured to display parameter range line graphs on a PCA pump user interface screen in accordance with the lower and upper limits established for a user-inputted parameter. Additionally, the example methods, systems, and apparatus are configured to generate and display user alerts when a user inputted parameter value affects the range of values acceptable by a previously entered parameter value. The disclosed systems, methods, and apparatus prevent human programming errors by minimizing the need for extensive user interaction with PCA pumps and the need to toggle between programming screens. Further, the disclosed systems, methods, and apparatus prevent human programming errors by displaying the dynamic relationship between different programming parameters. As such, the disclosed systems, methods, and apparatus increase efficient infusion administration and prevent programming errors that might result in patient discomfort or injury.

In light of the disclosure herein, and without limiting the scope of the invention in any way, in a first aspect of the present disclosure, which may be combined with any other aspect listed herein unless specified otherwise, a PCA pump includes an actuator, a syringe holder, a display screen, a processor, and a memory storing instructions, which when executed by the processor, cause the processor to receive a user-selected infusion therapy type and a user-inputted parameter value, receive the lower and upper limits of the parameter type associated with the user-inputted parameter value and user-selected infusion therapy type, compare the user-inputted parameter value to the lower and upper limits of the parameter type, generate a graph on the display screen that shows the lower and upper limits of the parameter, and generate a graphic for the user-inputted parameter value based on where the user-inputted parameter value falls within the lower and upper limits of the parameter type.

In a second aspect of the present disclosure, which may be combined with any other aspect listed herein unless specified otherwise, the upper and lower limits of the parameter type define an infusion therapy type acceptable range.

In a third aspect of the present disclosure, which may be combined with any other aspect listed herein unless specified otherwise, the graph is a line bar graph.

In a fourth aspect of the present disclosure, which may be combined with any other aspect listed herein unless specified otherwise, the graphic is positioned on the graph.

In a fifth aspect of the present disclosure, which may be combined with any other aspect listed herein unless specified otherwise, the position of the graphic on the graph corresponds with where the user-inputted parameter value falls within the infusion therapy type acceptable range.

In a sixth aspect of the present disclosure, which may be combined with any other aspect listed herein unless specified otherwise, a syringe volume sensor is configured to collect real time data on loaded syringe volume while programming the infusion parameters.

In a seventh aspect of the present disclosure, which may be combined with any other aspect listed herein unless specified otherwise, the memory stores instructions, which when executed by the processor, cause the processor to calculate the total syringe volume based on the user-selected infusion therapy type, compare the total syringe volume with the loaded syringe volume, and generate a user alert when total syringe volume exceeds loaded syringe volume.

In an eighth aspect of the present disclosure, which may be combined with any other aspect listed herein unless specified otherwise, the memory stores instructions, which when executed by the processor, cause the processor to calculate the total syringe volume based on the user-selected infusion therapy type, compare the total syringe volume with the loaded syringe volume, and generate a user alert when total syringe volume exceeds loaded syringe volume.

In a ninth aspect of the present disclosure, which may be combined with any other aspect listed herein unless specified otherwise, the memory stores instructions, which when executed by the processor, cause the processor to receive a user-inputted lockout interval value, calculate the PCA boluses allowed per hour, and display the PCA boluses allowed per hour.

In a tenth aspect of the present disclosure, which may be combined with any other aspect listed herein unless specified otherwise, the memory stores instructions, which when executed by the processor, cause the processor to receive a user-inputted PCA limit value, and limit the PCA boluses allowed per hour according to the PCA limit value.

In an eleventh aspect of the present disclosure, which may be combined with any other aspect listed herein unless specified otherwise, a method for operating a PCA pump based on user input includes selecting an infusion therapy type from a preloaded drug library displayed on a display screen, inputting a parameter value, transmitting the infusion therapy type selected and inputted parameter value to a processor, wherein the processor communicates with a memory and determines the lower and upper limits of the parameter type associated with the user-inputted parameter value and user-selected infusion therapy type, transmitting instructions from the memory to the processor for generating a lower and upper limit graphical display on the display screen, comparing the user-inputted parameter value to the instructions received by the processor, generating a graphical display on the display screen displaying the lower and upper limits of the parameter type in different colors, and generating a graphic for the user-inputted parameter value based on where the user-inputted parameter value falls within the lower and upper limits of the parameter type.

In a twelfth aspect of the present disclosure, which may be combined with any other aspect listed herein unless specified otherwise, the preloaded drug library includes information on the upper and lower limits of a parameter type, including the soft limits and hard limits.

In a thirteenth aspect of the present disclosure, which may be combined with any other aspect listed herein unless specified otherwise, the graphical display is a line bar graph and the graphic is positioned on the line bar graph.

In a fourteenth aspect of the present disclosure, which may be combined with any other aspect listed herein unless specified otherwise, the method includes calculating a total syringe volume based on the user-selected infusion therapy type, comparing the total syringe volume with a loaded syringe volume collected from a syringe volume sensor, and generating a user alert when total syringe volume exceeds loaded syringe volume.

In a fifteenth aspect of the present disclosure, which may be combined with any other aspect listed herein unless specified otherwise, the method includes receiving a user-inputted lockout interval value, calculating PCA boluses allowed per hour, and displaying the PCA boluses allowed per hour.

In a sixteenth aspect of the present disclosure, which may be combined with any other aspect listed herein unless specified otherwise, the method includes receiving a user-inputted PCA limit value, and limiting the PCA boluses allowed per hour according to the PCA limit value.

In a seventeenth aspect of the present disclosure, which may be combined with any other aspect listed herein unless specified otherwise, a PCA pump system includes a network, a gateway, an Electronic Medical Records (EMR) server, and a PCA pump. The PCA pump is capable of delivering an intravenous infusion therapy to a patient via one or more intravenous (“IV”) line sets, based on inputs entered by a user, the PCA pump connects to the network, the gateway and the EMR server connect to the network, and the PCA pump is communicatively coupled to the gateway and the EMR server via the network.

In an eighteenth aspect of the present disclosure, which may be combined with any other aspect listed herein unless specified otherwise, the PCA pump comprises an actuator, a syringe holder, a display screen, a syringe volume sensor configured to collect real time data on loaded syringe volume, a processor, and a memory storing instructions, which when executed by the processor, cause the processor to receive a user-selected infusion therapy type and a user-inputted parameter value, receive the lower and upper limits of the parameter type associated with the user-inputted parameter value and user-selected infusion therapy type, compare the user-inputted parameter value to the lower and upper limits of the parameter type, generate a graph on the display screen that shows the lower and upper limits of the parameter, and generate a graphic for the user-inputted parameter value based on where the user-inputted parameter value falls within the lower and upper limits of the parameter type.

In a nineteenth aspect of the present disclosure, which may be combined with any other aspect listed herein unless specified otherwise, the memory stores instructions, which when executed by the processor, cause the processor to calculate the total syringe volume based on the user-selected infusion therapy type, compare the total syringe volume with the loaded syringe volume, generate a user alert when total syringe volume exceeds loaded syringe volume, receive a user-inputted lockout interval value, calculate the PCA boluses allowed per hour, and display the PCA boluses allowed per hour.

In a twentieth aspect of the present disclosure, which may be combined with any other aspect listed herein unless specified otherwise, the memory stores instructions, which when executed by the processor, cause the processor to receive a user-inputted lockout interval value, calculate the PCA boluses allowed per hour, display the PCA boluses allowed per hour, receive a user-inputted PCA limit value, and limit the PCA boluses allowed per hour according to the PCA limit value.

In a twenty-first aspect of the present disclosure, any of the structure, functionality, and alternatives disclosed in connection with any one or more of FIGS. 1 to 9 may be combined with any other structure, functionality, and alternatives disclosed in connection with any other one or more of FIGS. 1 to 9.

In light of the present disclosure and the above aspects, it is therefore an advantage of the present disclosure to minimize user interaction with the PCA pump control system to minimize human error.

It is another advantage of the present disclosure to provide a PCA pump user interface that visually aids the user during the programming process to ensure user-inputted parameters are within the lower and upper limits in relation to the infusion therapy type being administered and any interrelation with other parameters inputted by the user.

It is another advantage of the present disclosure to provide a PCA pump user interface that generates and displays a loaded syringe volume versus calculated syringe volume comparison.

It is another advantage of the present disclosure to provide a PCA pump user interface that displays lockout interval information.

It is another advantage of the present disclosure to provide a PCA pump user interface that allows the user to limit the amount of PCA boluses allowed, such that the amount of boluses allowed is lower than the maximum boluses calculated.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a system level diagram of a PCA pump within a hospital information system, according to an example embodiment of the present disclosure.

FIG. 2 is a perspective view of an example PCA pump comprising the Baxter® Novum pump, which may be included within the hospital system of FIG. 1, according to an example embodiment of the present disclosure.

FIG. 3 is a software component diagram of the on-screen guidance operations performed by the PCA pump of FIG. 1, according to an example embodiment of the present disclosure.

FIG. 4 is a diagram of the line graph range operations performed by the PCA pump of FIG. 1, according to an example embodiment of the present disclosure.

FIG. 5 is a diagram of the syringe volume comparison operations performed by the PCA pump of FIG. 1, according to an example embodiment of the present disclosure.

FIG. 6 is a diagram of the lockout interval calculation operations performed by the PCA pump of FIG. 1, according to an example embodiment of the present disclosure.

FIGS. 7A-7B are sample views of a user interface for on-screen parameter programming guidance for use in the display screen of the PCA pump of FIG. 1, according to an example embodiment of the present disclosure.

FIGS. 8 and 9 are consecutive views of a user interface for on-screen parameter programming guidance during use, according to an example embodiment of the present disclosure.

### DETAILED DESCRIPTION

Methods, systems, and apparatus are disclosed herein for a PCA pump user interface that provides on-screen parameter programming guidance to prevent dosage errors. The example methods, systems, and apparatus are configured to display parameter range line graphs on a PCA pump user interface screen in accordance with the lower and upper limits established for a user-inputted parameter. Additionally, the example methods, systems, and apparatus are configured to generate and display user alerts when a user inputted parameter value affects the range of values acceptable by a previously entered parameter value.

For on-screen parameter programming guidance, the user selects the drug being infused and enters a parameter value, and a processor and memory in the PCA pump communicate this information with each other. The processor matches the user-selected drug with an entry in a preloaded drug library. The entry from the preloaded drug library includes information on the upper and lower limits for the parameter inputted by the user. The processor finds the related lower and upper limit graphical instructions, and transmits them to the processor. The processor executes the instructions it receives from the memory, monitoring any changes in the user-inputted parameters, displaying a range line graph on the PCA pump screen, and changing the range line graphs for previously inputted parameter values as the user enters new parameter values. As such, there is no need for the user to toggle between screens when inputting a given parameter type to check changes to previously entered parameter values, as the PCA pump interface dynamically changes all range line graphs to guide the user in programming. The on-screen parameter programming guidance further displays the loaded syringe volume, while alerting the user if their prescribed therapy dose exceeds the total loaded syringe volume, so as to warn the user of the need for syringe replacement or refilling during infusion therapy. Finally, the on-screen parameter programming guidance displays the total PCA doses allowed per hour for a given therapy type, based on the user inputted PCA dose amount and time of interval lockout.

Reference is made herein to a memory. As disclosed herein, a memory refers to a device that holds electronic data and/or instructions for immediate use by a processor and/or pump control system. The memory is able to receive and transmit data.

Reference is made herein to a processor. As disclosed herein, a processor refers to a device that executes instructions stored by the memory. The memory receives and transmits data.

Reference is made herein to an infusion. As disclosed herein, an infusion refers to the putting of fluids, intravenously through the use of a needle or catheter, into the bloodstream. The fluids can be a drug, supplement, or a mix thereof.

Reference is made herein to a drug library. As disclosed herein, a drug library refers to an indexed list of drugs and supplements. Each entry contains the name of the substance (both scientific and branded names), important parameters like maximum and minimum dosages, concentration information, infusion rates, and whether the drug is administered with or without PCA boluses. It should be noted that additional information can be included in the entries for a drug library.

While the example methods, apparatus, and systems are disclosed herein as operating with PCA pumps, it should be appreciated that the methods, apparatus, and systems may be operable with other pumps. For example, the methods, apparatus, and systems may provide for on-screen parameter programming guidance to prevent dosage errors in syringe pumps based on user-inputted parameters and commands.

### Medical Environment Embodiment

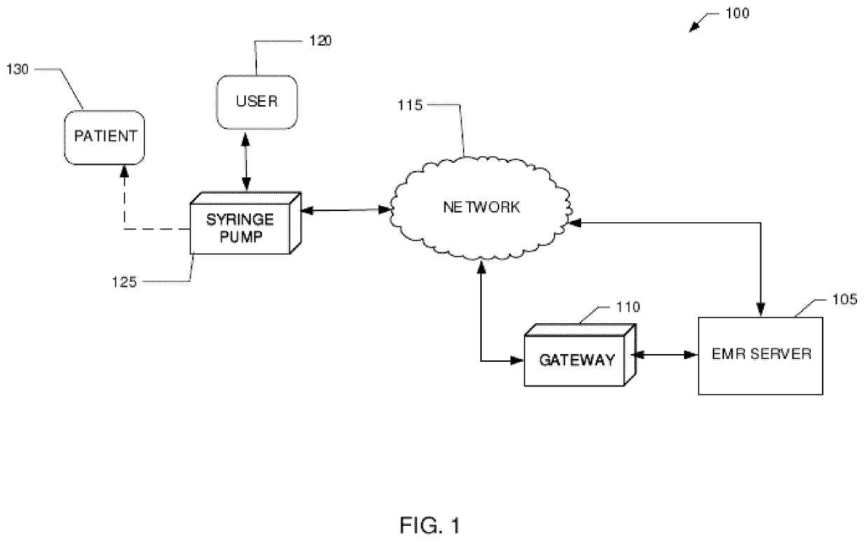

FIG. 1 is a system level diagram of a PCA pump within a hospital information system 100. The example system 100 includes a PCA pump 125, a network 115, a gateway 110, and an Electronic Medical Records (“EMR”) server 105. The PCA pump 125 is capable of delivering an intravenous infusion therapy to a patient 130 via one or more intravenous (“IV”) line sets, based on inputs entered by the user 120. The PCA pump 125 connects to the network 115. In addition, the gateway 110 and EMR server 105 also connect to the network 115. As such, the PCA pump 125 is communicatively coupled to the gateway 110 and EMR server 105 via the network 115. In some embodiments, multiple PCA pumps 125 connect to the network 115, the gateway 110, and the EMR server 105. It must be noted that these connections may be wireless, such as via Bluetooth®, or wired, via a serial, Ethernet, CAN, or USB connection.

The gateway 110 is configured to receive infusion therapy type data (e.g., drug name, syringe volume, and bolus volume when applicable) from the PCA pump 125, and route the data to an EMR server 105. In some embodiments, the gateway 110 is configured to convert the data from, for example, EXTCOM message(s) to HL7 message(s). In yet other embodiments, the network 115 and the gateway 110 are omitted from the system 100.

The gateway 110 may also be configured to transmit operating parameters or prescription parameters to the PCA pump 125. For example, the gateway 110 may send an electronic prescription (or software update) to the PCA pump 125 at a predetermined time and/or when the PCA pump 125 is available to accept the prescription. In other instances, the PCA pump 125 may be configured to periodically poll the gateway 110 to determine if an electronic prescription (or software update) is awaiting to be downloaded to the pump.

Relatedly, the PCA pump 125 transmits infusion therapy progress data to the network 110. The network 110 then converts the therapy progress data to a protocol for transmission via Ethernet to the gateway 110 via the network 115. The gateway 110 may include, for example, the Baxter® IQ Enterprise® gateway. As such, the gateway 110 may be configured to integrate with the EMR server 105 or other hospital system to facilitate the transmission of the infusion therapy progress data from the PCA pump 125 to, for example, an EMR related to the patient 130.

In one embodiment, the EMR server 105 is also communicatively coupled to a pharmacy server (not shown), which is configured to create and/or transmit medication orders corresponding to, for example, a prepared medication (not shown). A medication order includes an electronic record or entry, which identifies a patient (e.g., a patient identifier) and infusion parameters for administration. The medication order is assigned a unique identifier. In some embodiments, the medication order may be printed on a label attached to a medication container that is fluidly coupled to the PCA pump 125. The medication order itself associates a patient identifier with a medication identifier. The EMR server 105 is configured to use the patient identifier in the medication order to store or otherwise associate the medication order with a patient's EMR.

In an alternate embodiment, the system 100 may also include a clinician device (not shown; e.g., a smartphone, tablet computer, laptop computer, workstation, etc.) such that the user 120 could monitor patient data.

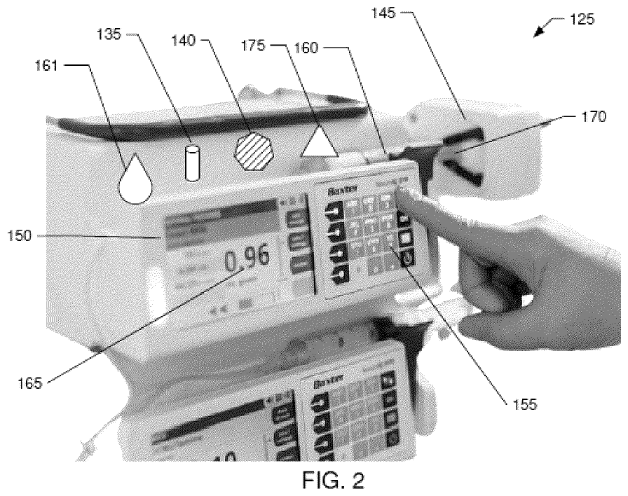

FIG. 2 is a perspective view of an example PCA pump 125. The illustrated infusion pump 125 is the Baxter® Novum IQ PCA pump. In this embodiment, the PCA pump 125 includes a display 150 with interfaces 145 and a keypad 155 to enable a clinician to specify or program an infusion therapy or graphical display command. The PCA pump 125 uses a motor connected to an actuator 170 to actuate a plunger 165 within a syringe 160. Moreover, the PCA pump 125 includes a syringe volume sensor 161 configured to measure real-time syringe volume.

The PCA pump 125 also includes a memory 135 and a processor 140. The memory 135 stores one or more drug libraries, like Dose IQ, that include particular program parameter limits based on care area, dose change, rate of change, drug name, concentration, patient age, patient weight, etc. The limits are configured to ensure that a received prescription or entered infusion therapy is within acceptable ranges and/or limits decided by a medical facility, doctor, or clinician. The drug libraries also include information as to whether an infusion therapy includes PCA boluses, or administers at a continuous constant rate without PCA boluses.

The memory 135 also stores patient history data which may include, but is not limited to, a table of different infusion therapy intervals (1-, 2-, 4-, 8-, 12-, 24-hour, and cumulative), Loading Dose (LD) & Clinician Bolus (CB) Bolus, PCA Bolus, Programmed Intermittent Epidural Boluses (PIEB), Continuous infusion rate (ml/hr), and Total Volume (ml/hr). The memory 135 continuously stores real-time patient history data.

The processor 140 is configured to execute machine-readable instructions stored in the memory 135. Execution of the machine-readable instructions by the processor 140 causes the PCA pump 125 to perform the operations described herein.

As noted previously, the PCA pump 125 is connected to and communicates with the gateway 110 (FIG. 1) via the network 115 (FIG. 1). As also noted, the PCA pump 125 is configured to monitor the progress of the infusion therapy and periodically transmit infusion therapy progress data (e.g., medical device data) to the gateway 110 (FIG. 1). The therapy progress data, as disclosed herein, may include, for example, an infusion rate, a dose, a total volume infused, a time remaining for the therapy, a medication concentration, rate change, a volume remaining within a medication container, a medication name, a patient identifier, titration information, bolus information, a care area identifier, a timestamp when the data was generated, an alarm condition, an alert condition, an event, etc. The PCA pump 125 may transmit the data continuously, periodically (e.g., every 30 seconds, 1 minute, etc.), or upon request by the gateway 110 (FIG. 1).

In some embodiments, the PCA pump 125 may also be communicatively coupled to one or more physiological sensors. For example, the PCA pump 125 may be connected to a pulse oximetry sensor, a blood pressure cuff, an access disconnection device, and/or a weight scale. The PCA pump 125 may be configured to integrate or otherwise include, for example, data from the pulse oximetry sensor into the therapy progress data or, alternatively, transmit the pulse oximetry data separately to the gateway 110 (FIG. 1). The gateway 110 FIG. 1) can then access the EMR server 105 (FIG. 1) to record this data in a patient's electronic medical record.

### On-Screen Parameter Programming Guidance

As previously noted, at the start of an infusion therapy protocol, the user inputs the infusion therapy type, namely the drug name, into the PCA pump 125. After the user enters the drug name into the PCA pump 125, they must program the various parameters (i.e. the PCA dose amount or the lockout interval).

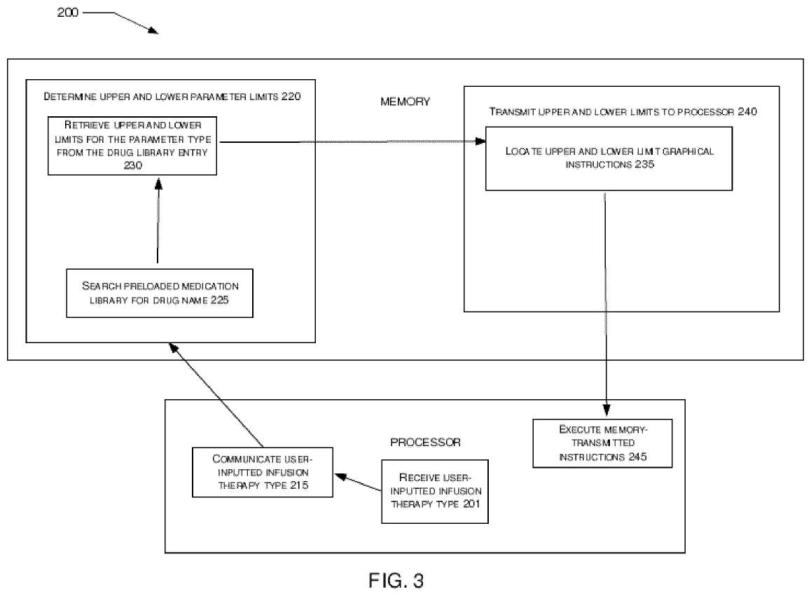

FIG. 3 is a software component diagram of the operations 200 performed by the PCA pump of FIG. 1 to display on-screen parameter programming guidance based on user input in relation to the drug name initially entered by the user. As seen in the diagram in box 201, first the processor 140 receives the user-inputted drug name and parameters (i.e. the PCA dose amount or the lockout interval) data. The parameter is received in the form of value such as a dosage and the drug name is received in the form of a unique identifier. The processor 140 then transmits data containing the user-inputted drug name unique identifier and parameter value 215 to the memory 135. The memory 135 in conjunction with the processor 140 receives unique identifier and searches the drug library for a drug library entry with that matches the unique identifier entered by the user 225. More specifically, the memory 135 in conjunction with the processor 140 indexes its drug library, comparing the drug library entries to the drug or supplement identifier it received.

Upon finding a match, the processor 140 determines the lower and upper limits, including hard and soft limits, of the parameter entered by the user, as it relates to drug name entered by the user 220. More specifically, the processor 140 accesses the matching drug library entry and retrieves data on the upper and lower limits of the parameter entered by the user. The processor 140 then locates upper and lower limit graphical instructions associated with the parameter entered by the user 235, in the memory 135. The memory 135 in conjunction with the processor 140 then transmits the instructions to the processor 140.

The processor 140 receives the memory-transmitted instructions and executes the instructions (block 245). To implement the instructions, the processor 140 compares the parameter value entered by the user to the upper and lower limits it received in the memory-transmitted instructions. The processor 140 does two things concurrently: it generates a range line graph to display on the user interface, and it also locks the entry of other parameters in the PCA pump 125 if the parameter value entered by the user exceeds the upper limits it received in the memory-transmitted instructions.

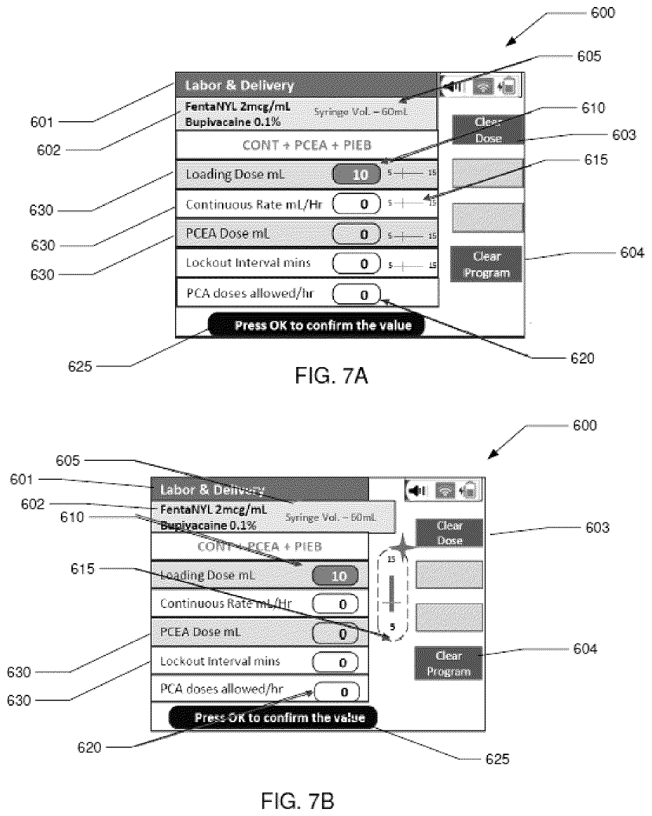

To generate the range line graph, the processor 140 receives the memory-transmitted instructions and generates a graphic that corresponds with the lower and upper limits for the parameter entered by the user. More specifically, the graphic is, for example, a line graph displayed next to the therapy type and the parameter value entered by the user (see FIGS. 7A-9).

Concurrently, the processor 140 compares the parameter value entered by the user to previously entered parameter values. If the parameter value entered by the user counteracts with any previously entered parameter value, the processor locks the entry of other parameters into the PCA pump 125 to prevent the user from administering the therapy. However, the user can enter a subsequent parameter value. Once the subsequent parameter value is within the lower and upper limits for the parameter entered by the user, and the subsequent parameter value does not cause counteractions with any previously entered parameter, the processor 140 unlocks the PCA pump 125 to allow the user to administer the therapy. It should be noted that the processor continues to compare the parameter value(s) entered by the user as the user changes the parameter value(s).

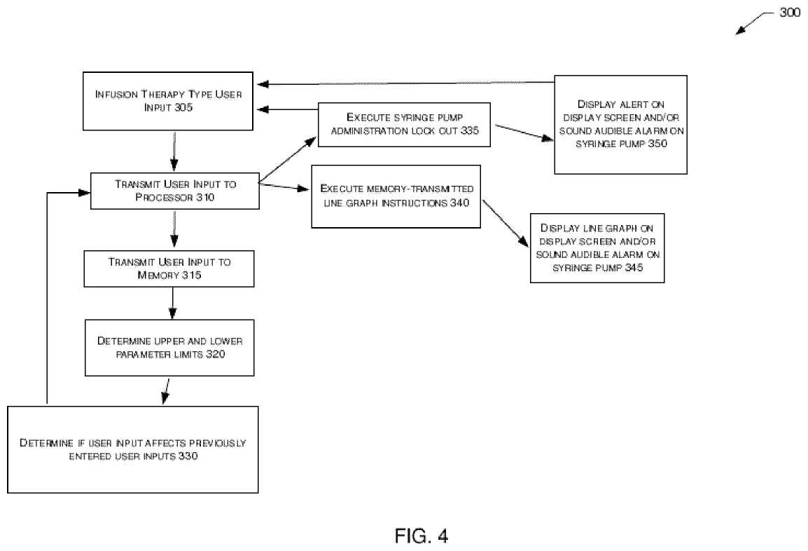

FIG. 4 shows a diagram of a pump control system process for on-screen parameter programming guidance based on user input. As seen in the diagram, the user inputs their desired drug name and parameter value, such as the PCA dose amount or the lockout interval (block 305). The user does this by entering, for example, a drug name (an infusion therapy type) and numerical dosage using a keypad and screen display on the PCA pump.

The data corresponding with the user input, a unique drug name and parameter value, is then transmitted to the processor (block 310). Once it arrives at the processor, the processor transmits the user input data to the memory (block 315).

Upon arrival, the memory in conjunction with the processor receives the user input data, locates the unique drug name identifier, and accesses its preloaded drug library to find a matching drug name entry. Upon locating a match, the memory in conjunction with the processor retrieves data associated with the matching entry. The memory in conjunction with the processor then determines the upper and lower limit graphical instructions associated with the parameter value entered by the user (block 320). To achieve this, the memory in conjunction with the processor retrieves from the matching drug name entry, the data indicating lower and upper parameter limits (block 320). More specifically, the lower and upper parameter limits are in the form of soft and hard limits. Soft limits refer to lower or upper limits that are set in the drug library and can be overridden by the user. On the other hand, hard limits refer to lower or upper limits that are set in the drug library and that cannot be overridden by the user.

After this, the memory in conjunction with the processor determines if the parameter value entered in the user counteracts with any previously entered parameter values (block 330). If the parameter value entered counteracts with any previously entered parameter values, the memory in conjunction with the processor then retrieves and transmits PCA pump lock out instructions (block 335). In some embodiments, the PCA pump may further display an alert message on the screen and/or sound an audible alarm (block 350). The PCA pump will remain locked and displaying an alert message and/or sounding an audible alarm until the user enters a subsequent parameter that does not counteract previously entered parameters. In effect, if the user enters a different, new parameter that is interrelated with a previously entered parameter value(s), the processor determines how, if at all, the new parameter affects the lower and upper limits of the previously entered parameter(s) and accordingly

Conversely, if the parameter value entered does not counteract with any previously entered parameter values, the memory in conjunction with the processor then retrieves and transmits line graph instructions (block 340). More specifically, the processor generates a range line graph, for example, a bar graph horizontally displayed next to each parameter (see FIGS. 7A-9).

### Syringe Volume

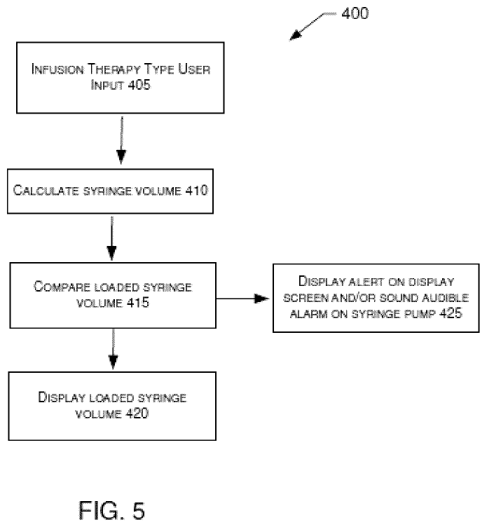

FIG. 5 shows a diagram of a pump control system process for displaying syringe volume based on user input. As seen in the diagram, the user inputs their desired drug name and parameter value in the form of prescribed dose (block 405). After this, the PCA pump calculates the syringe volume in accordance with the prescribed dose (block 410). Specifically, the user input, the drug name and prescribed dose, is transmitted to the processor which then the transmits it to the memory. Upon arrival, the memory in conjunction with the processor receives the user input data, locates the unique drug name identifier, and accesses its preloaded drug library to find a matching drug name entry. Upon locating a match, the memory in conjunction with the processor retrieves data associated with the matching entry. The memory in conjunction with the processor then calculates the total syringe value based on the prescribed dose. Concurrently, the processor receives loaded syringe volume data from the syringe volume sensor 161.

After this, the processor compares the loaded syringe volume with the calculated syringe volume (block 415). Finally, the memory in conjunction with the processor then retrieves and transmits instructions to display the loaded syringe volume on the display screen (block 420). Specifically, if the calculated syringe volume is less than or equal to the loaded syringe volume, then the PCA pump displays the loaded syringe volume on the display screen. However, in alternate embodiments, if the calculated syringe volume is greater than or equal to the loaded syringe volume, the memory in conjunction with the processor then retrieves and transmits instructions to display an alert message on the screen and/or sound an audible alarm (block 425). As such, the user is made aware that their prescribed dose will require additional syringe replacement and/or refilling.

### Lockout Intervals

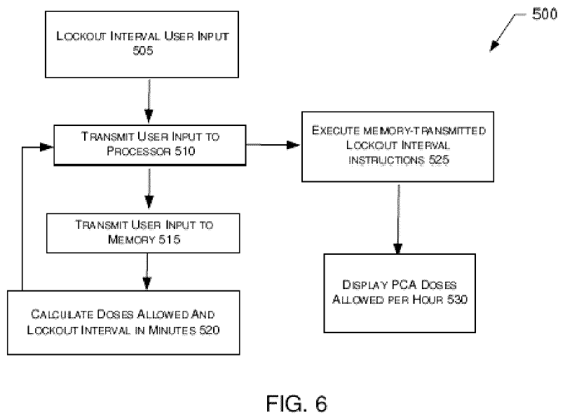

FIG. 6 shows a diagram of a pump control system process for calculating lockout intervals based on user input. As seen in the diagram, the user inputs their desired lockout interval for a given therapy type, such as a specific drug (block 505). The user does this by entering, for example, a PCA dose in mL and lockout interval in minutes, using a keypad and screen display on the PCA pump. The user can also enter, at this point, a restriction on the number of PCA boluses the PCA pump can administer for a given prescribed dose. In other words, the user can limit the maximum number of PCA boluses a patient can self-administer.

The data corresponding with the user input, PCA dose in mL and lockout interval in minutes, is then transmitted to the processor (block 510). Once it arrives at the processor, the processor transmits the user input data to the memory (block 515).

Upon arrival, the memory in conjunction with the processor receives the user input data, locates the unique drug name identifier, and accesses its preloaded drug library to find a matching drug name entry. Upon locating a match, the memory in conjunction with the processor retrieves data associated with the matching entry. The memory in conjunction with the processor then calculates the total PCA doses allowed for the duration of a given therapy, based on the upper and lower limits for the therapy type (specific drug), the total prescribed drug dosage, and the lockout interval in minutes entered by the user (block 520).

After this, the processor receives the memory-transmitted instructions and executes the instructions (block 525). Specifically, the processor executes lockout interval instructions and it displays the PCA doses allowed per hour on PCA pump (blocks 525 and 530).

If the user lowers the lockout interval or increases the PCA dose, such that the change in these parameters will lead to overdose, as determined by the processor in conjunction with the preloaded drug library, the PCA pump will generate and display an alert message and, in some embodiments, play an audible alarm. Relatedly, the user can enter a limited bolus input on the PCA pump which further limits the number of boluses the PCA pump can administer for a given prescribed dose. In that context, the user can prevent overdosing for patients that are opioid naïve, opioid tolerant, or pediatric, by affirmatively programming the PCA pump to reduce the number of PCA boluses for a given therapy (specific drug) being administered.

### Graphical User Interface

FIGS. 7A to 9 show sample views of a user interface for on-screen parameter programming guidance for use in the display screen of the PCA pump of FIG. 1.

As seen in FIG. 7A, the on-screen parameter programming guidance user interface 600 shows the drug name of the infusion therapy 602, the care area 601, and loaded syringe volume 605. The interface 600 also shows the various parameters 630 that the user can input when programming the PCA pump. By displaying the various parameters 630, the user interface 600 helps prevent programming errors. Specifically, the user is able to see the various parameters 630 in a single view without having to toggle between screens while also attempting to remember what values they entered for each of the various parameters 630. This is important because in the medical field, and specifically in the context of infusion therapy administration, if the user makes a programming error, the error could lead to patient injury or death. For example, as previously noted, certain patients, like pediatric or opioid naïve patients, are especially vulnerable in cases of programming errors. Again, by displaying the various parameters 630 in a single screen, the user interface 600 prevents programming errors by displaying information in a format that is comprehensive and salient for the user.

Each parameter 630 includes a user input field 610 that displays the user inputted parameter value. Relatedly, the interface 600 further includes a value confirmation button 625, a clear dosage button 603, and a clear program button 604. Importantly, the interface 600 displays derived parameter values 620, such as PCA doses allowed per hour. Displaying derived parameter values 620 allows the user to see and understand the dynamic relationship between the various parameters 630 and make real time adjustments. For example, if a derived parameter value 620 is dangerous, the user will see this in real time and adjust the various parameters 630 until they achieve a desired derived parameter value 620. Alternate embodiments can include different buttons such as, but not limited to, a patient history or edit therapy button.

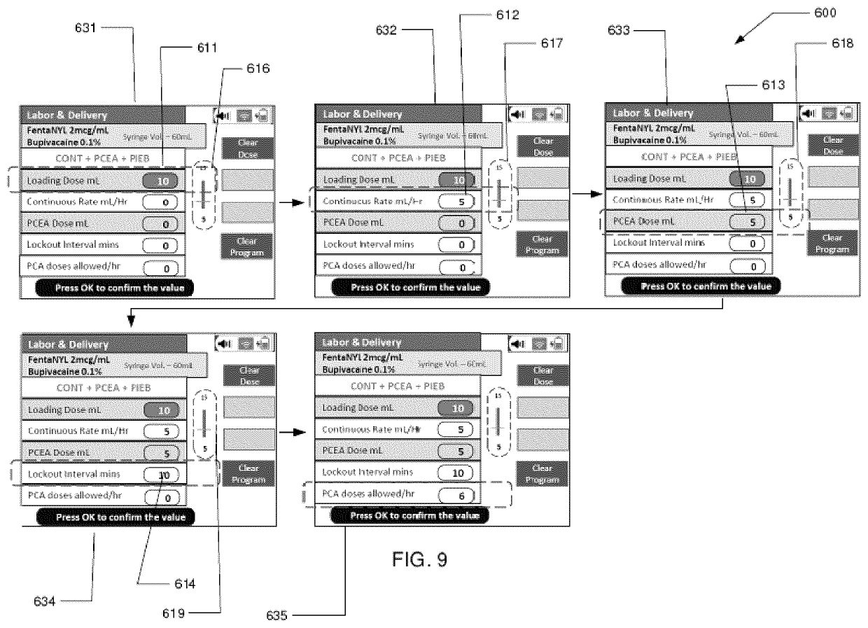

Notably, the user interface shows a range line graph 615, which corresponds with the lower and upper limits for a given parameter. As seen in FIG. 7A, the range line graph 615 includes numbers on opposite ends of a line, where the numbers correspond with the lower and upper limits of the given parameter. Moreover, the range line graph 615 may include a small dash line that corresponds with where user inputted parameter value falls between the lower and upper limits of the given parameter. While in FIG. 7A an individual range line graph 615 is displayed next to each parameter 630, as seen in FIG. 7B, alternate embodiments include an interface 600 with a single range line graph 615 that corresponds with whichever parameter 630 the user selects. In the case of FIG. 7B, the user interface 600 displays only the range line graph 615 for the parameter 630 that the user is currently entering. However, in the embodiment shown on FIG. 7B, the user can still select other previously entered parameters 630 and, when the user does this, the respective range line graph 615 is displayed. As a result, the user still has access to all the range line graphs 615 for all the parameters 630 in one user interface 630. This is shown in FIG. 9 and further explained below.

Importantly, the individual range line graph 615 for each parameter 630 is also dynamic. Specifically, as the user programs each parameter 630, the range line graphs 615 for any previously programmed parameters 630 also update to reflect how any subsequently programmed parameter values affect any previously programmed parameters 630. As such, once again, the user interface 600 helps the user avoid programming errors by displaying range line graphs 615 that are dynamic in nature and represent the interrelation between the various parameters 630, and, importantly, how programming the various parameters 630 affects previously programmed parameters 630. In turn, once again, the user does not have to toggle between screens, and this avoids programming errors.

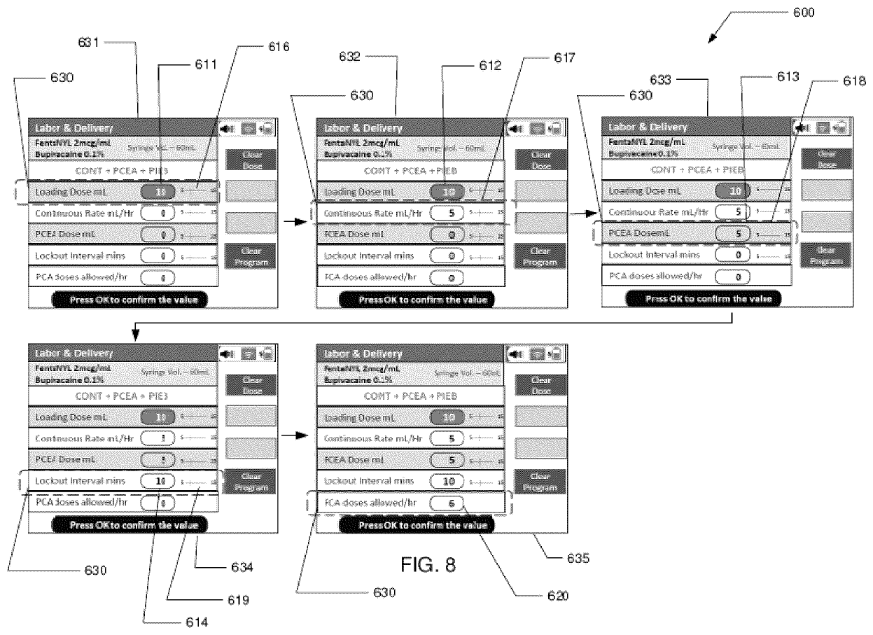

Finally, FIGS. 8 and 9 show further views of sample views of a user interface for on-screen parameter programming guidance for use in the display screen of the PCA pump of FIG. 1. Specifically, FIG. 8 shows consecutive views of the user interface on-screen parameter programming guidance 600 during use, corresponding with a display design from FIG. 7A with multiple range line graphs 615 each located next to each parameter 630.

As seen in FIG. 8, at the start of programming 631, the user enters a first user input 611 and this generates a first range line graph 616. As the user continues programming, as seen in subsequent entries shown in 632 to 634, the user enters subsequent user inputs 612 to 614. As the user enters subsequent user inputs 612 to 614, the user interface 600 concurrently displays range line graphs 617 to 619. Notably, each of the range line graphs 617 to 619 dynamically change as each additional subsequent user input 612 to 614 is entered. Finally, at the end of programming 635, the user is able to see the derived parameter value 620. As explained above, the user can the change any previously programmed parameters 630 (i.e. change user inputs 611 to 614) to achieve their desired derived parameter value 620.

Similarly, FIG. 9 shows consecutive views of the user interface on-screen parameter programming guidance 600 during use, corresponding with a display design from FIG. 7B with a single range line graph that corresponds with whichever parameter 630 the user selects. The difference between FIG. 9 and FIG. 8 is the fact that the user interface 600 in FIG. 9 displays a single range line graph. Specifically, as seen in FIG. 9, during programming 631 to 635, the user enters user inputs 611 to 614. As the user enters a given user input, for example user input 612, the user interface 600 displays only the corresponding range line graph, for example range line graph 617 that corresponds with user input 612. The user can see the range line graph (i.e. 616 to 619) by selecting a parameter 630 and associated user input (i.e. 611 to 614). As such, once again, the user has access to dynamic visual information showing the interrelation between the various parameters 630, and, importantly, how programming the various parameters 630 affects previously programmed parameters 630. In turn, once again, the user does not have to toggle between screens, and this avoids programming errors.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A PCA pump comprising:
an actuator;
a syringe holder;
a display screen;
a processor; and
a memory storing instructions, which when executed by the processor, cause the processor to
receive a user-selected infusion therapy type and a user-inputted parameter value,
receive the lower and upper limits of the parameter type associated with the user-inputted parameter value and user-selected infusion therapy type,
compare the user-inputted parameter value to the lower and upper limits of the parameter type,
generate a graph on the display screen that shows the lower and upper limits of the parameter, and
generate a graphic for the user-inputted parameter value based on where the user-inputted parameter value falls within the lower and upper limits of the parameter type.

2. The PCA pump of claim 1, wherein the upper and lower limits of the parameter type define an infusion therapy type acceptable range.

3. The PCA pump of claim 2, wherein the graph is a line bar graph.

4. The PCA pump of claim 3, wherein the graphic is positioned on the graph.

5. The PCA pump of claim 4, wherein the position of the graphic on the graph corresponds with where the user-inputted parameter value falls within the infusion therapy type acceptable range.

6. The PCA pump of claim 1, further comprising a syringe volume sensor configured to collect real time data on loaded syringe volume.

7. The PCA pump of claim 6, further comprising memory storing instructions, which when executed by the processor, cause the processor to calculate the total syringe volume based on the user-selected infusion therapy type, compare the total syringe volume with the loaded syringe volume, and generate a user alert when total syringe volume exceeds loaded syringe volume.

8. The PCA pump of claim 1, further comprising memory storing instructions, which when executed by the processor, cause the processor to calculate the total syringe volume based on the user-selected infusion therapy type, compare the total syringe volume with the loaded syringe volume, and generate a user alert when total syringe volume exceeds loaded syringe volume.

9. The PCA pump of claim 1, further comprising memory storing instructions, which when executed by the processor, cause the processor to receive a user-inputted lockout interval value, calculate the PCA boluses allowed per hour, and display the PCA boluses allowed per hour.

10. The PCA pump of claim 9, further comprising memory storing instructions, which when executed by the processor, cause the processor to receive a user-inputted PCA limit value, and limit the PCA boluses allowed per hour according to the PCA limit value.

11. A method for operating a PCA pump based on user input comprising:
selecting an infusion therapy type from a preloaded drug library displayed on a display screen;
inputting a parameter value;
transmitting the infusion therapy type selected and inputted parameter value to a processor, wherein the processor communicates with a memory and determines the lower and upper limits of the parameter type associated with the user-inputted parameter value and user-selected infusion therapy type;
transmitting instructions from the memory to the processor for generating a lower and upper limit graphical display on the display screen;
comparing the user-inputted parameter value to the instructions received by the processor;
generating a graphical display on the display screen displaying the lower and upper limits of the parameter type in different colors; and
generating a graphic for the user-inputted parameter value based on where the user-inputted parameter value falls within the lower and upper limits of the parameter type.

12. The method of claim 11, wherein the preloaded drug library includes information on the upper and lower limits of a parameter type, including the soft limits and hard limits.

13. The method of claim 11, wherein the graphical display is a line bar graph and the graphic is positioned on the line bar graph.

14. The method of claim 11, further comprising calculating a total syringe volume based on the user-selected infusion therapy type, comparing the total syringe volume with a loaded syringe volume collected from a syringe volume sensor, and generating a user alert when total syringe volume exceeds loaded syringe volume.

15. The method of claim 11, further comprising receiving a user-inputted lockout interval value, calculating PCA boluses allowed per hour, and displaying the PCA boluses allowed per hour.

16. The method of claim 15, further comprising receiving a user-inputted PCA limit value, and limiting the PCA boluses allowed per hour according to the PCA limit value.

17. A PCA pump system comprising:
a network;
a gateway;
an Electric Medical Records (EMR) server; and
a PCA pump;
wherein the PCA pump is capable of delivering an intravenous infusion therapy to a patient via one or more intravenous (“IV”) line sets, based on inputs entered by a user;
wherein the PCA pump connects to the network;
wherein the gateway and the EMR server connect to the network; and
wherein the PCA pump is communicatively coupled to the gateway and the EMR server via the network.

18. The PCA pump system of claim 17, wherein the PCA pump comprises:
an actuator;
a syringe holder;
a display screen;
a syringe volume sensor configured to collect real time data on loaded syringe volume;
a processor; and
a memory storing instructions, which when executed by the processor, cause the processor to
receive a user-selected infusion therapy type and a user-inputted parameter value,
receive the lower and upper limits of the parameter type associated with the user-inputted parameter value and user-selected infusion therapy type,
compare the user-inputted parameter value to the lower and upper limits of the parameter type,
generate a graph on the display screen that shows the lower and upper limits of the parameter, and
generate a graphic for the user-inputted parameter value based on where the user-inputted parameter value falls within the lower and upper limits of the parameter type.

19. The PCA pump system of claim 18, further comprising memory storing instructions, which when executed by the processor, cause the processor to calculate the total syringe volume based on the user-selected infusion therapy type, compare the total syringe volume with the loaded syringe volume, generate a user alert when total syringe volume exceeds loaded syringe volume, receive a user-inputted lockout interval value, calculate the PCA boluses allowed per hour, and display the PCA boluses allowed per hour.

20. The PCA pump system of claim 18, further comprising memory storing instructions, which when executed by the processor, cause the processor to receive a user-inputted lockout interval value, calculate the PCA boluses allowed per hour, display the PCA boluses allowed per hour, receive a user-inputted PCA limit value, and limit the PCA boluses allowed per hour according to the PCA limit value
